(19) United States
(12) Patent Application Publication
(10) Pub. No.: US 2017/0156639 A1
(43) Pub. Date: Jun. 8, 2017

(54) Posture mapping and posture monitoring system and method

(71) Applicant: Gokhale Method Enterprise, Stanford, CA (US)

(72) Inventors: Esther Gokhale, Stanford, CA (US); Mark Leavitt, North Plains, OR (US); Rachel Dreilinger, Beavercreek, OR (US); Björn Krüger, Bonn (DE); Jonas Müller, Bonn (DE)

(73) Assignee: Gokhale Method Enterprise, Stanford, CA (US)

(21) Appl. No.: 15/530,457

(22) Filed: Jan. 17, 2017

Related U.S. Application Data
(63) Continuation-in-part of application No. 13/987,537, filed on Aug. 5, 2013, now abandoned.
(60) Provisional application No. 62/497,100, filed on Nov. 9, 2016.
(60) Provisional application No. 61/850,950, filed on Feb. 28, 2013.

(51) Int. Cl. A61B 5/11; A61B 5/00

(52) U.S. Cl. CPC A61B 5/1116; A61B 5/0004; A61B 5/742; A61B 5/486; A61B 5/0022; A61B 5/6823; A61B 5/0059; A61B 5/1128; A61B 2562/0219

(57) ABSTRACT

A system for and method of mapping and monitoring posture disclosed. The system includes sensor units that independently collect posture data corresponding to body locations or body positions on subjects and generate posture signals. The posture signals are wirelessly transmitted to one or more computers that are used to process the posture signals and generate posture feedback of the subjects in real-time. The sensor units are preferably accelerometer sensor units. Sensor units are assigned or learn the body locations or body positions, classify body locations or body positions, map and monitor posture, assign posture or activities of subjects and transmit the posture signals to the one or more computers by low-power radio transmitters to provide posture feedback. The system collectively is adaptive for mapping and monitoring posture from body locations or body positions corresponding to spines, arms, legs, necks, heads, thighs, hips, feet and hands of the subjects.

### RELATED APPLICATIONS

This application is a continuation-in-part of co-pending U.S. patent application Ser. No. 13/987,537, filed on Aug. 5, 2013, and titled “POSTURE MONITORING SYSTEM”, which claims priority under 35 U.S.C. §119(e) from the Co-pending U.S. Provisional Patent Application Ser. No. 61/850,950, filed on Feb. 28, 2013, and titled “POSTURE DETECTION SYSTEM”. The U.S. patent application Ser. No. 13/987,537, filed on Aug. 5, 2013, and titled “POSTURE MONITORING SYSTEM”, and the Co-pending U.S. Provisional Patent Application Ser. No. 61/850,950, filed on Feb. 28, 2013, and titled “POSTURE DETECTION SYSTEM” are both hereby incorporated by reference.

This application also claims priority under 35 U.S.C. §119(e) from the Co-pending U.S. Provisional Patent Application Ser. No. 63/497,100, filed on Nov. 9, 2016, and titled “POSTURE MONITORING SYSTEM”, the contents of which are also hereby incorporated by reference.

### FIELD OF THE INVENTION

This invention relates to systems for and methods of mapping posture and monitoring posture. More specifically, this invention relates to systems and methods that use sensor technology to monitor body movement and body positions of human subjects to provide posture feedback to help maximize optimal posture for improved health and/or therapy while performing activities.

### BACKGROUND OF THE INVENTION

Posture has been linked to a person's overall wellness or health. While there are some body positions that are considered to represent optimized posture, body positions often need to be customized for a particular persons's situation. Further, proper range of motion and body positions often need to be customized to meet a person's therapeutic needs while preforming one or more activities.

Achieving and maintaining good or proper posture often requires changes in behavior, habits and lifestyle. Achieving good posture is often a process, with optimized posture being the end goal. In order to achieve this goal, it is useful to have systems that map and monitor posture dynamically, continuously and/or in real-time.

### SUMMARY OF THE INVENTION

The present invention is directed to systems and methods that use sensor technology to monitor posture of individuals, hereafter subjects, and provide posture feedback of the subjects. The posture feedback is provided directly to the subjects or to a data-base that can be viewed by the subjects and/or healthcare providers.

Posture, herein, refers to body positions and body movements. Posture, herein refers to static body positions while performing sedentary activities, such sitting or sleeping and to dynamic body positions and body movements while performing activities, such as running or swimming. The present invention is used to monitor overall posture of subjects or postures as it relates body positions or body locations of sensors on the subjects. For example, the system and method are used to monitor postures from spines, arms, legs, necks, heads, thighs, hips, feet and hands of the subjects, or any combinations thereof.

Posture feedback is provided to encourage subjects to progress towards a posture goal for improving wellness, health and/or therapeutic results while performing activities. The posture goal may, or may not, represent what is considered to be an optimal posture because posture goals often need to be tailored to a particular situation or need of a subject. For example, posture goals for a subject with a spinal injury will be different than posture goals for a subject with a healthy spine. Also, posture goals for a subject undergoing physical therapy from a significant arm, leg or hip injury will be different than posture goals for a subject without a significant arm, leg or hip injury.

The sensor technology that is used in the system and method of the present invention includes pressure or contact sensors, photo sensors, optical or camera sensors, flex sensors, accelerometers or any combination thereof. In operation, the sensors provide body location data, body position data and/or range of motion data, hereafter posture data, of a subject. The posture data is transmitted as posture signals to a computer system. The computer system then provides feedback to the subject and/or healthcare monitoring system based on a posture protocol that compares and analyzes the posture signals relative to a posture goal or predicted posture signals.

The computer system preferably includes a server or central computer that is accessible over the internet and/or a cellular network from a personal computer, a laptop computer, a tablet and/or a smart phone, hereafter, mobile internet enabled device. The server or central computer includes a processor and memory for running programs that compare and analyze the posture signals sent from sensor units that are attached at or near body locations or positions on a subject.

In operation, the mobile internet enabled device acquires the posture signals and transmits the posture signals to the server or central computer over a network (internet or cellular). The posture signals can be sent as raw posture data or the posture signals can be analyzed or partially analyzed and processed on the mobile internet enabled device. Regardless of where the posture signals are analyzed and processed, the posture signals are used to provided posture feedback. It will be clear to one skilled in the art that any number of intermediary computing devices can be used to run software that analyzes and processes posture signals to generate posture information that is then used to provide posture feedback. A server or central computer, a mobile internet enabled device or any intermediary computing devices is also referred to herein as computing unit.

Posture feedback can take a number of forms. For example, posture feedback can be tactile feedback, audio feedback, visual feedback, or any combination thereof. Preferably, posture feedback is provided in the form of a graphical representation of the subjects posture relative to a posture goal. The graphical representation is preferably accessible or viewable by the subject via one or more mobile internet enabled device with a suitable display screen.

Sensors are generally integrated into a housing with a wireless transmitter. A sensor, a housing, a transmitter, a battery and any other required electrical components is collectively referred to herein as a sensor unit. Multiple sensor units used in the system and method of the present invention are referred to, herein, as a sensor structure. A sensor structure or a portion of the sensor structure with multiple sensors units coupled together is referred to, herein, as a sensor module. Sensor structures and sensor modules of the present invention have several sensing zones. The sensing zones, sensors and/or sensor units can be integrated into wearable article, such as an article of clothing and/or include multiple detached sensors and/or sensor units that are configured to be positioned at or near various body locations or body positions of a subject.

Where a portion the sensor structure or a sensor module is integrated in to an wearable article, the article is, but not limited to, a belt, a vest, a band, a brace, sportswear, footwear or combinations thereof. In operation, portions of the sensor structure, such as a sensor module and/or sensor units, are attached to skin of the subject through a tape and/or adhesive or are held against or near skin of the subject via the wearable article.

As described above, the system and method of the present invention use sensors to map, monitor and/measure posture. The sensors are preferably accelerometer sensors. Alternative embodiments of the invention the system and method use contact sensors and/or pressure sensors, alone or in combination with accelerometer sensors. Contact sensors include, but are not limited to, flex sensors, stretch sensors and pressure sensors. Flex sensors, also known as bend sensors, are uni-directional and bi-directional. In general, all flex sensors change resistance when bent or deformed in one or more directions. Pressure sensors usually include a piezo-resistive materials. For example, a pressure sensor includes a film with a carbon-impregnated polyolefin fiber that is laminated between a highly conductive, thin and flexible textile, such as Copper-polyester taffeta fabric and/or a Nickel-copper shielding material. Further details about contact sensors and pressure sensors are provided in the U.S. patent application Ser. No. 13/987,537, filed on Aug. 5, 2013, and titled “POSTURE MONITORING SYSTEM”, the contents of which are incorporated by reference.

The sensor units that are used in the present invention include a wireless transmitter and electrical connections that allow the sensor units to communicate with a computing unit. The wireless transmitter is preferably a radio transmitter that transmits low-power radio signals over a peer-to peer network, such as a bluetooth network. The computing unit preferably includes a radio receiver or transducer for receiving posture signals from each of sensor units independently.

### BRIEF DESCRIPTION OF THE DRAWINGS

FIG. 1A illustrates a block-flow diagram outlining the system for and the method of mapping and monitoring posture, in accordance with the embodiments of the invention.

FIG. 1B illustrates a sensor unit, in accordance with the embodiments of the invention.

FIG. 2 illustrates a high level-block-flow diagram for analyzing posture signals from multiple posture sensors, in accordance with the embodiments of the invention.

FIG. 3A illustrates a sensor module that charges multiple sensor batteries used for mapping and monitoring posture, in accordance with the embodiments of the invention.

FIG. 3B illustrates a sensor placement template for spacing multiple sensors on a subject, in accordance with the embodiments of the invention.

FIG. 3C illustrates applying multiple sensors to along a spine of a subject using the sensor placement template shown in FIG. 3B, in accordance with the embodiments of the invention.

FIG. 3D illustrates multiple sensors positioned along a spine of a subject for mapping and monitoring posture, in accordance with the invention.

FIGS. 4A-C illustrate configurations for positioning multiple sensors on a subject for mapping and monitoring posture, in accordance with the embodiments of the invention.

FIGS. 5A-C illustrate posture feedback graphical screen-shot representations corresponding to different spinal positions of a subject, in accordance with the invention.

FIGS. 6A-B illustrate positioning multiple sensors along appendages of a subject for mapping and monitoring posture, in accordance with the invention.

### DETAILED DESCRIPTION OF THE INVENTION

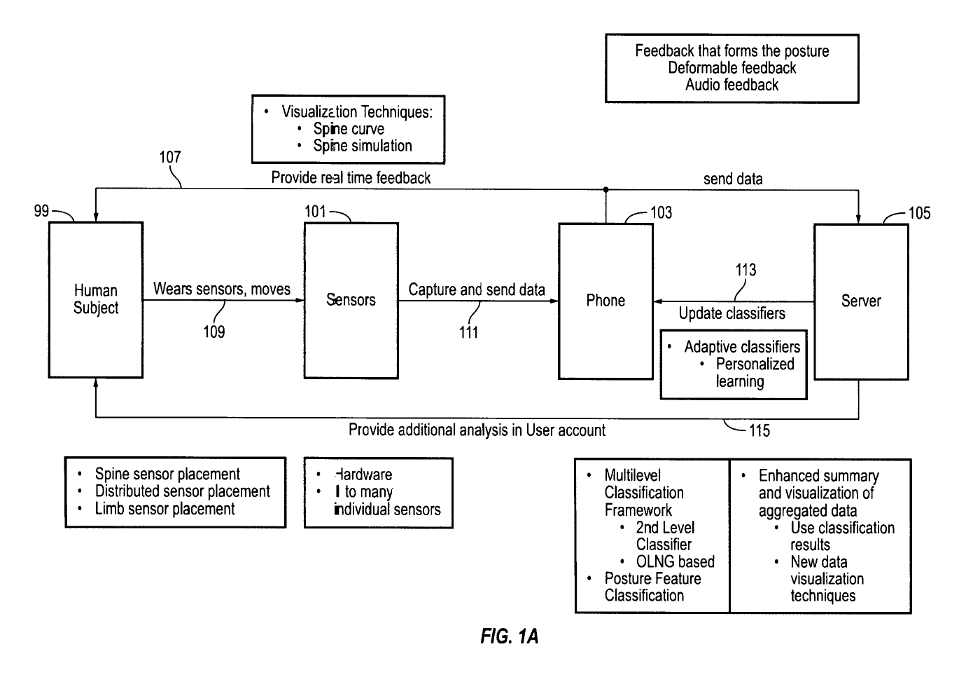

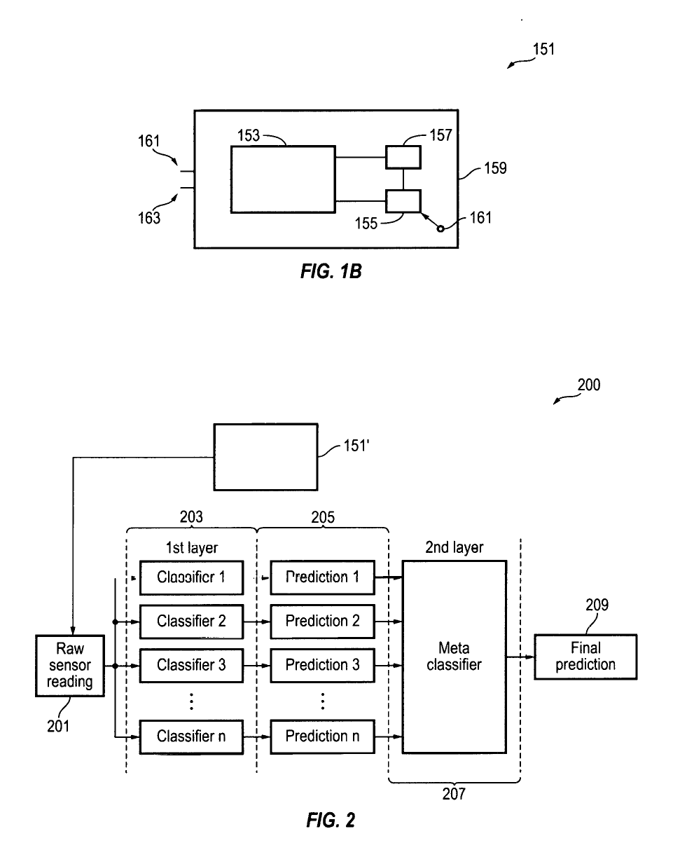

The present invention is directed to a system for and a method of mapping and monitoring posture. FIG. 1A illustrates block-flow diagram outlining the system for and the method of mapping and monitoring posture. The system of the present invention includes sensors units 101 for generating posture data. The sensors units 101 include any number and types of sensors, such as described above, but preferably include multiple accelerometer sensors.

Referring to FIG. 1B, each accelerometer sensor 153 is preferably contained within a housing 159. The accelerometer sensor 153, the housing 159 and any other electrical component contained within the housing 159 or attached to the housing 159 are collectively referred to herein as a sensor unit 151. The sensor unit 151 also has a radio transmitter or transducer 155 for transmitting low-power radio signals (posture signals) to a mobile internet enabled device 103, such as a smart phone (FIG. 1A) over a peer-to peer network. The sensor unit 151 also include a battery 157 for powering the transmitter or transducer 155 and connectors 161 and 163 for charging the battery 157. The sensor unit 151 can also include a micro-processor for processing raw posture data generated by a accelerometer sensor 153 and instructing the transmitter or transducer 155 when and how to transmit posture signals to the mobile internet enabled device 103.

Referring back to FIG. 1A, the sensor units 101, such as the sensor unit 151 (FIG. 1B) are attached or coupled to a subject, referred to as sensor locations. The sensor locations can correspond to any number of body locations or body positions or combinations of body locations or body positions on the subject. In operation, the sensors 153 continuously generate posture data corresponding the respective body locations or body positions on the subject and the transmitters or transducers 155 continuously transmitted posture signals to the mobile internet enabled device or computer unit 103 in real-time, as indicated by the arrow 111. The posture signals are stored in memory of the mobile internet enabled device or computer unit 103 and are then analyzed by software running on the mobile internet enabled device or computer unit 103 to provide posture feedback, indicated by the arrow 107.

Alternatively or in addition to the mobile internet enabled device 103 analyzing the posture signals and providing posture feedback, the posture signals are transmitted to a server or central computer 105, indicated by the arrow 107, through the internet, where the posture signals are stored in memory on the server or central computer 105 and analyzed by software running on a processor of the server or central computer 105. In this application, the mobile internet enabled device or computer unit 103 is acting as internet transmitter to relay the posture signals to the server or central computer 105.

The server or central computer 105 then can provide posture feedback to the subject 99 over the internet as indicated by the arrow 115, or via the mobile internet enabled device 103, as indicated by the arrow 113. It will be clear to one skilled in the art, that various portions of the analysis of posture signals transmitted from the sensor units 101 or 151 (FIG. 1B) can take place between the mobile internet enabled device 103 and the server or central computer 105, or between any number of intermediary computing devices. Where large qualities of posture data and posture signals are compiled or collected, analysis of the posture signals on the server or central computer 105 is preferred. The server or central computer 105 can be used to store posture information and provide support for personal posture accounts to any number of subjects. In operation, the server or central computer 105 supports web-pages that allow subjects to log into their posture account and view posture information, posture feedback and/or allow authorized administrators to analyze posture information or posture signals collected from multiple subjects.

Sensor units 101 or 151 (FIG. 1B) are assigned body locations or body positions on a subject 99 using any number of suitable methods. For example, sensor units 101 or 151 (FIG. 1B) can transmit a radio signal that provides an address that corresponds to an assigned body location or body position of the subject 99. For example, sensor units 101 or 151 (FIG. 1B) can be assigned one through four, corresponding to ascending locations or positions along a spine of the subject 99, body locations or body positions along an appendage of the subject 99 and/or any combination thereof. Alternatively, sensor units 101 or 151 (FIG. 1B) can include input selectors 161 (FIG. 1B) that allows the assigned body position on the subject 99 to be selected and or changed. In yet further embodiments of the invention, the sensor units 101 or 151 (FIG. 1B) are intelligent sensor units 101 or 151 (FIG. 1B). Intelligent sensor units 101 or 151 (FIG. 1B) transmit samples of posture signals while in a learning mode. Based on an analysis of the samples of posture signals via the mobile internet enabled device 103 and/or the server or central computer 105, the sensors units 101 or 151 (FIG. 1B) are assigns body locations or body positions on the subject 99. Regardless of how the sensor units 101 or 151 (FIG. 1B) are assigned to body locations or body positions on the subject, the sensor units 101 or 151 (FIG. 1B) can be configured to use machine learning techniques and/or algorithms to better classify body locations or body positions, map and monitor posture, assign posture or activities of subjects and provide more accurate posture feedback.

FIG. 2 shows a high-level method block-diagram 200 for analyzing or predicting posture from posture signals collected from multiple posture sensor units 151′, such as the sensor units 101 (FIG. 1A) or 151 (FIG. 1B). In operation the raw sensor signals 201 are collected from the sensors 151′. In the step 203 the raw sensor signals 201 are classified. For example, posture signals 201 are classified for potential movements, locations, activities and etc to generate classified posture signals 203. The classified posture signals 205 are then matched statistically to a predicted or expected posture signals in 205. If the predicted or expected posture signals 205 match, the posture signal 201, they are assigned a classification and analyzed in a meta-classifier layer 207. In the meta-classifier layer 207, the classified signals 205 are collectively analyzed with each other or against each other to generate a final posture prediction 209. For example, if a subject is sitting, but moving his or her arms. The arms may be assigned to a running activity and the legs may be assigned to a standing activity. While the running activity and the sitting activity may match the predicted posture signals corresponding to running and standing, the subject is not running and the subject is not standing still. In the meta-classifier layer 207, the classified posture signals 205 are collectively analyzed to generate a posture prediction 209 corresponding to the subject lifting weights with his or her arms. As described above, the system of the present invention can use machine learning techniques and/or algorithms to better classify body locations or body positions, map and monitor posture, assign posture or activities of subjects and provide more accurate posture feedback.

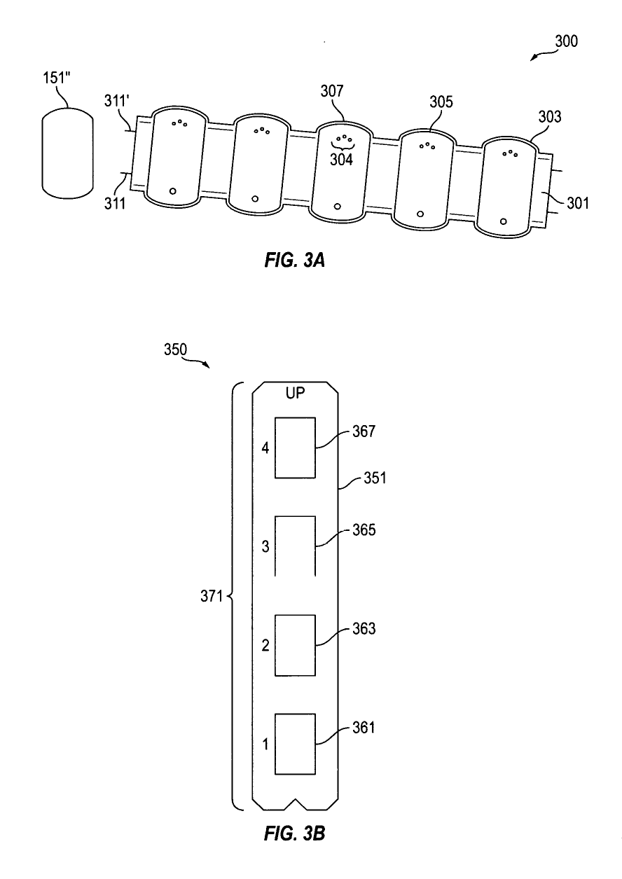

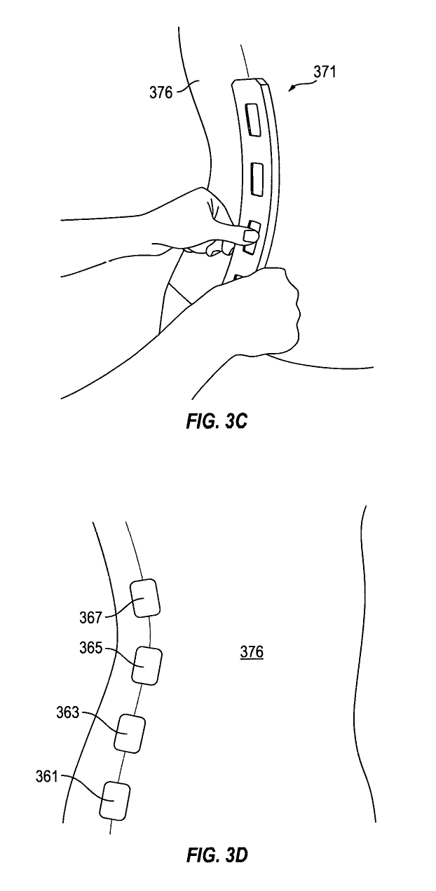

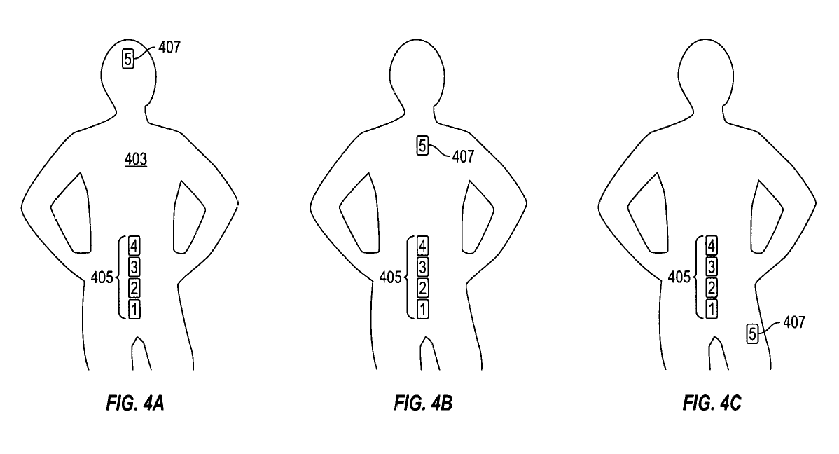

FIG. 3A illustrates a view 300 of a sensor module 301 for holding multiple sensor units 151″, such as 151 (FIG. 1B) for mapping and monitoring posture. The sensor module 301 has several cradles or slot features 303, 305 and 307 for holding the multiple sensor units 151″ and connectors 304 for electrical coupling the multiple sensor units 151″ to the sensor module 301. The sensor module includes connectors 311 and 311′ for electrically coupling the sensor module 301 to a charger and/or a computer (not shown).

In operation the sensor units 151″ are placed in cradles or slot features 303, 305 and 307 of the posture module 301 and sensor unit batteries 157 (FIG. 1B) are charged through the connectors 304. The connectors are powered through the connectors 311 and 311 that electrically couple to a charging unit (not shown). The sensor units 151″ are placed against or near assigned body locations or body positions of a subject. The sensor units 151″ are assigned body locations or body positions body of the subject using any of the techniques described above. The sensor units 151″ then generate posture signals that are transmitted by a radio transmitter or transducer 155 (FIG. 1B) to a mobile internet enabled device 103 and/or a server or a central computer 105 (FIG. 1A).

FIG. 3B shows a view 350 of a sensor placement template 371 with a sensor template 351 and multiple sensors 361, 363, 365 and 367. Referring to FIG. 3C, in operation the sensor placement template 371 is placed along a body location of a subject 367, in this case along a spine of the subject. The sensor template 351 is then removed leaving the sensors 361, 363, 365 and 367 attached along the body location, as shown in FIG. 3D. It will be clear to one skilled in the art that the sensor units can be placed at any number of body location and/or body positions or combinations thereof.

FIG. 4A-C show alternative body locations or positions of sensors units 405 and 407. In FIG. 4A, a strip of sensor units or sensor module 405 is placed along a spine of a subject 403. In addition, a sensor unit 407 is placed on or near a head of the subject 403. In FIG. 4B, a strip of sensor units or sensor module 405 is placed along a spine of a subject 403. In addition, a sensor unit 407 is placed on or near a neck or collar bone area of the subject 403. In FIG. 4C, a strip of sensor units or sensor module 405 is placed along a spine of a subject 403 and a sensor unit 407 is placed on or near a hip or thigh area of the subject 403. In operation the strip of sensor units or sensor module 405 and the sensor unit 407 act collectively to generate posture signals that are used to provide posture feedback. The strip of sensor units or sensor module 405 are coupled together through a module structure 301, or are uncoupled, such as shown in FIG. 3D. The sensor units 405/407 used are preferable sensor units, similar to the sensor unit 151 described with reference to FIG. 1B.

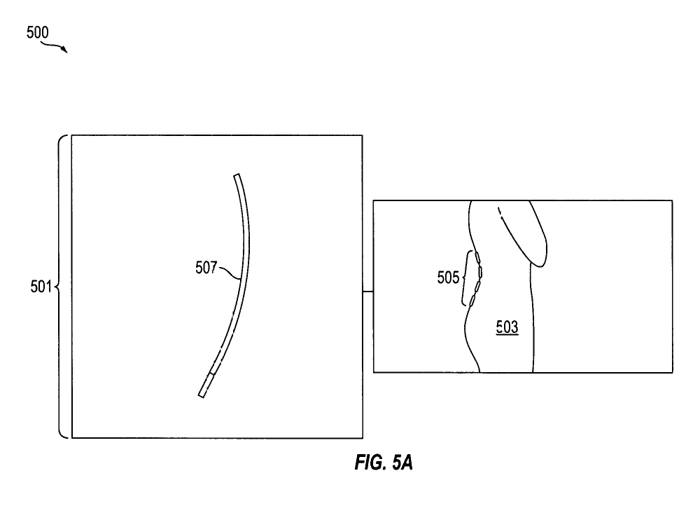

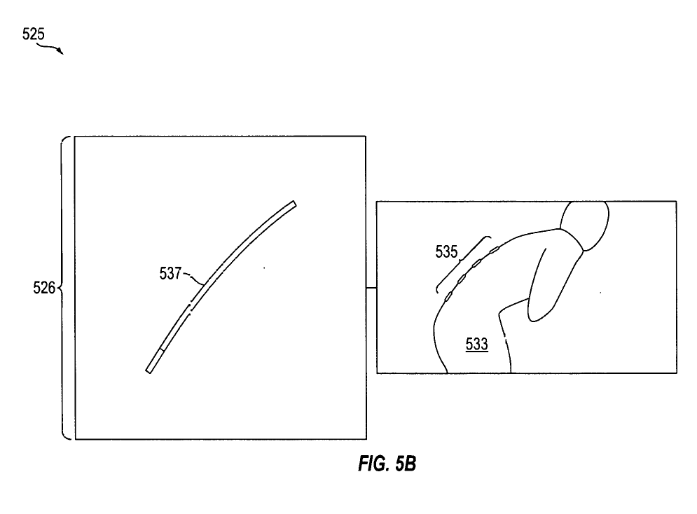

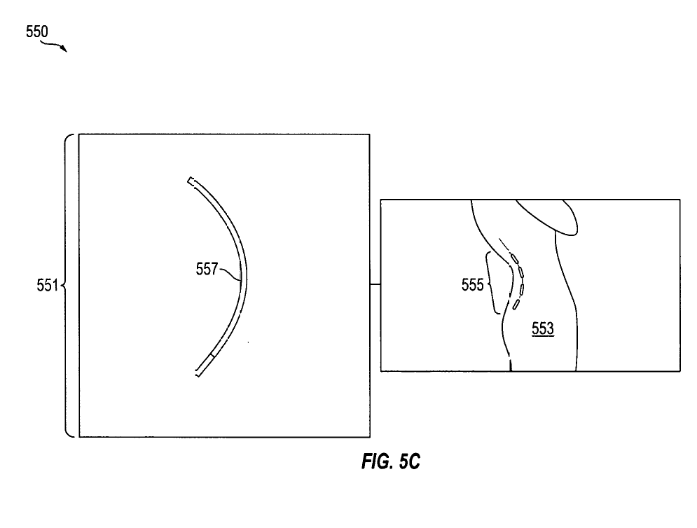

FIG. 5A-C show views 500, 525 and 550, respectively, of arrays or sets or sensor units, such as sensor units 151 (FIG. 1B), sensor units 151′ (FIG. 2) and sensor units 151″ (FIG. 3A) and corresponding graphical feedback representations of a subjects posture. In FIG. 5A, a subject 503 is standing straight with an array or set of sensor units 505 placed along a spine of the subject 503. On a corresponding screen shot 501 on a suitable computer or display (not shown), the set of sensor units 505 is used to generate a line 507 that represents curvature of the spine. The screen-shot 501 can also include a comparison line that represents an ideal curvature of the spine to further provide the subject 503 with posture feedback. In FIG. 5B, the subject 533 is leaning forward with the array or the set of sensor units 535 placed along the spine of the subject 533. On a corresponding screen-shot 526 on the computer or display, the set of sensor units 535 is used to generate a line 537 that represents curvature of the spine. In FIG. 5C, the subject 553 is leaning backwards with the array or the set of sensor units 555 placed along the spine of the subject 553. On a corresponding screen-shot 551 on the computer or display, the set of sensor units 555 is used to generate a line 557 that represents curvature of the spine.

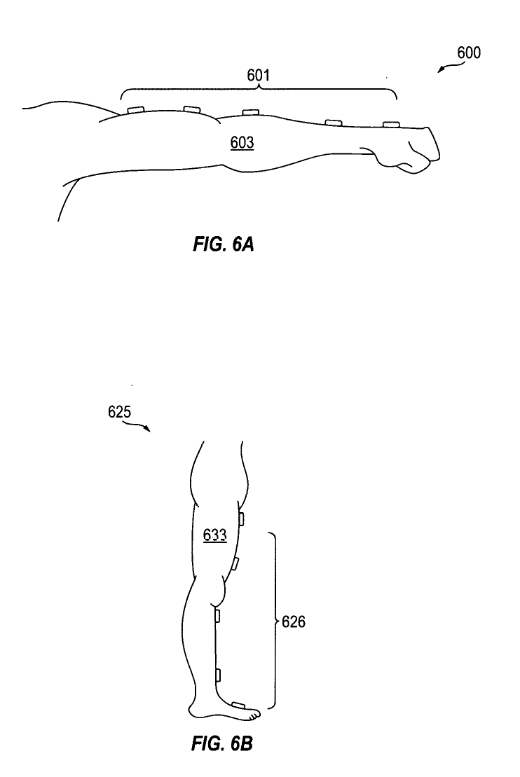

FIGS. 6A-B show views 600 and 625, respectively, of arrays or sets or sensor units 601 and 626, such as sensor units 151 (FIG. 1B), sensor units 151′ (FIG. 2) and sensor units 151″ (FIG. 3A) placed along an arm 603 of a subject and a leg 633 a subject. In accordance with the embodiments of the invention, the arrays or sets or sensor units 601 and 626 are used alone or in combination with others arrays or sets or sensor units, such as described with reference to FIGS. 4A-C and FIGS. 5A-C, to dynamically monitor posture of the subject in real time.

The present invention has been described in terms of specific embodiments incorporating details to facilitate the understanding of the principles of construction and operation of the invention. As such, references, herein to specific embodiments and details thereof are not intended to limit the scope of the claims appended hereto. It will be apparent to those skilled in the art that modifications can be made in the embodiments chosen for illustration without departing from the spirit and scope of the invention. For example, sensor units can be configured to communicate with each other through low-power radio signals, run application firmware, include user interfaces to change settings and/or include displays to indicate modes of operation. Also, while sensor units have been mostly shown herein as being placed along a spine or appendages of a subject, sensor units can be placed at any body location or body position or combinations of body locations or body positions of the subject to map and monitor posture of the subject.

What is claimed is:

1. A system for mapping and monitoring posture comprising:
a) a plurality of sensor units for placing in contact communication with a subject, wherein the sensor units each have a sensor and a transmitter, wherein sensor units operate in a data collection mode to independently collect posture data corresponding to a body location of each of the sensor units on the subject, generate posture signals and wirelessly transmit the posture signals; and
b) a computer with a wireless receiver that communicates with each of sensor units and receives the posture signals from each of the sensor units, the computing unit further including a processor and memory for running software that analyzes the posture signals to generate posture feedback of the subject in real-time.

2. The system of claim 1, further comprising a sensor module for charging the plurality of sensor units.

3. The system of claim 2, wherein the sensor module includes electrical contacts for charging plurality of sensor units from the sensor module.

4. The system of claim 1, wherein the sensor is an accelerometer sensor.

5. The system of claim 1, wherein the wireless transmitter of the sensor unit and the wireless receiver of the computer is a low-power radio transmitter and a low-power radio receiver, respectively.

6. The system of claim 5, wherein the computer is a smart phone.

7. The system of claim 1, wherein the sensor units are assigned the body location on the subject.

8. The system of claim 7, wherein the sensor units operate in learning mode to assign the body location on the subject or classify posture of the subject.

9. The system of claim 1, further compering a display, wherein the posture feedback of the subject in real-time is a graphical representation generated by the computer on the display.

10. A method of mapping and monitoring posture comprising:
a) placing a plurality of sensor units corresponding to different body locations of a subject, wherein each of the plurality of sensor units includes a sensor for independently generating posture data and a transmitter for wirelessly transmitting posture signals generated from the posture data; and
b) wirelessly collecting the posture signals from a computer with a receiver and analyzing the posture signals to generate posture feedback of the subject in real-time.

11. The method of claim 10, wherein a portion of the sensor units are placed in a sensor template.

12. The method of claim 11, further comprising classify activity of the subject based on a portion of posture signals.

13. The method of claim 10, wherein the sensor is an accelerometer sensor.

14. The method of claim 10, wherein the transmitter of the sensor unit and the receiver of the computer is a low-power radio transmitter and a low-power radio receiver, respectively.

15. The method of claim 10, wherein the computer is a smart phone.

16. The method of claim 10, further comprising assigning the sensor units the different body location on the subject.

17. The method of claim 10, wherein assigning the sensor units includes collecting a sample of the posture signals any analyzing the sample of the posture signals against predicted posture signals.

18. The system of claim 10, further comprising displaying a graphical representation generated by the computer to provide the posture feedback of the subject.

19. The method of claim 10, wherein the different body locations of the subject include at least one appendage of the subject.

20. A system for mapping and monitoring posture comprising:
a) a plurality of sensor units for placing in contact communication with a subject, wherein the sensor units each have a sensor and a transmitter, wherein sensor units operate in a data collection mode to independently collect posture data corresponding to a body location of each of the sensor units on the subject, generate posture signals and wirelessly transmit the posture signals;
b) a mobile internet enabled device with a wireless receiver that communicates with each of sensor units and receives the posture signals from each of the sensor units; and
c) a central computer for receiving the posture signals from the mobile internet enabled device, the central computer including a processor and memory for running software that analyzes the posture signals generate posture feedback of the subject from the posture signals received and wherein the posture feedback is accessible from the mobile internet enabled device.
